(19) United States
(12) Patent Application Publication
(10) Pub. No.: US 2017/0332980 A1
(43) Pub. Date: Nov. 23, 2017

(54) APPARATUS AND METHOD FOR MONITORING HYPOGLYCAEMIA CONDITION

(71) Applicant: Firefly Health Pty Ltd, South Yarra, Victoria (AU)

(72) Inventors: Warwick Lewis Fifield, Melbourne (AU); Michael John Smith, Melbourne (AU); Samuel Thomas Bolton, Melbourne (AU); Jefferson Grey Harcourt, Melbourne (AU); Allison Kathryn Love, Melbourne (AU); Robert Harry Distel, Melbourne (AU); Stewart Grant Hamilton, Melbourne (AU)

(21) Appl. No.: 15/532,181

(22) PCT Filed: Dec. 1, 2015
(86) PCT No.: PCT/AU2015/050756

§ 371 Date: Jun. 1, 2017

(30) Foreign Application Priority Data
Dec. 2, 2014 (AU) 2014904878

(51) Int. Cl. A61B 5/00; A61B 5/01; A61B 5/145; A61B 5/11

(52) U.S. Cl. CPC A61B 5/7282; A61B 5/6826; A61B 5/1101; A61B 5/6843; A61B 5/01; A61B 5/6824; A61B 5/14532; A61B 2560/0242; A61B 2562/0219

(57) ABSTRACT

An apparatus to monitor for hypoglycaemia in a human, the apparatus including: two or more sensors to concurrently measure two or more physical properties over time, the physical properties including one or more temperatures and/or one or more movements of an arm and/or a hand of the human; and an electronic system configured to: receive signals representative of the measured physical properties; and process the received signals to generate an output indicative of hypoglycaemia in the human.

### RELATED APPLICATION

The originally filed specification of the following related application is incorporated by reference herein in its entirety: Australian Provisional Patent Application No. 2014904878, filed on 2 Dec. 2014 in the name of Firefly Health Pty Ltd.

### BACKGROUND

Diabetes mellitus is a medical condition resulting in chronic hyperglycaemia. Where exogenous insulin is needed to reduce blood glucose levels, hypoglycaemia is a common side-effect. If insufficiently controlled, hypoglycaemia can cause unconsciousness, seizure and death. A typical method for monitoring blood glucose levels is by frequent finger prick blood analyses, but this is intrusive, uncomfortable, sometimes inaccurate, inconvenient while active, and particularly inconvenient during sleep-time. Studies have found that, in Type 1 diabetic children, about half of severe hypoglycaemic episodes occur at night, and it is further estimated that as many as 1 in 10 patients with Type 1 diabetes die as a result of hypoglycaemia. Accordingly, there is a significant unmet need for improved hypoglycaemia monitoring technology to address this issue.

Available technology for monitoring diabetes, and alerting to dangerous events, is invasive, highly inconvenient and/or inaccurate. Other diseases and conditions may also require improved monitoring and alerting technologies.

It is desired to address or at least ameliorate one or more disadvantages in the prior art, or to at least provide a useful alternative.

### SUMMARY

In accordance with the present invention there is provided an apparatus to monitor for hypoglycaemia in a human, the apparatus including:

two or more sensors to concurrently measure two or more physical properties over time, the physical properties including one or more temperatures and/or one or more movements of an arm and/or a hand of the human; and

an electronic system configured to:
receive signals representative of the measured physical properties; and
process the received signals to generate an output indicative of hypoglycaemia in the human.

The present invention also provides a method to monitor for hypoglycaemia in a human, the method including:

concurrently measuring two or more physical properties over time, the physical properties including one or more temperatures and/or one or more movements of an arm and/or a hand of the human;

receiving signals representative of the measured physical properties; and

processing the received signals to generate an output indicative of hypoglycaemia in the human.

### BRIEF DESCRIPTION OF THE DRAWINGS

Some embodiments of the present invention are hereinafter further described, by way of non-limiting example only, with reference to the accompanying drawings, in which:

FIG. 1 is a sketch of an apparatus including a wrist assembly and a finger assembly;

FIG. 2A is a cross-sectional diagram of the wrist assembly;

FIG. 2B is a perspective view of one end of the wrist assembly;

FIG. 2C is a perspective view of the opposite end of the wrist assembly from the view of FIG. 2B;

FIG. 2D is a perspective view of the underside of the wrist assembly;

FIG. 2E is an exploded view of the wrist assembly;

FIG. 3A is a top-side perspective view of a printed circuit board assembly (PCBA) of the wrist assembly in an unassembled state;

FIG. 3B is an underside perspective view of the PCBA in FIG. 3A in the unassembled state;

FIG. 3C is a top-side perspective view of the PCBA in FIG. 3A in an assembled state;

FIG. 3D is an underside perspective view of the PCBA in FIG. 3A the assembled state;

FIG. 4A is an exploded view of the finger assembly;

FIG. 4B is a top-side perspective view of a distal end of the finger assembly;

FIG. 4C is a perspective view of a proximal end of the finger assembly;

FIG. 4D is a cross-sectional view of the finger assembly;

FIG. 4E is a top-side perspective view of a PCBA of the finger assembly in an unassembled state;

FIGS. 4F and 4G are perspective views of the PCBA of FIG. 4E in an assembled state;

FIG. 5 is a block diagram of electronic components of the apparatus;

FIG. 6 is a block diagram showing connections of a multifunctional connector of the apparatus;

FIG. 7 is a block diagram of operational modules in the apparatus;

FIG. 8 is a state diagram of operational modes of the apparatus;

FIG. 9 is a flowchart of a switching method performed by the multifunctional connector;

FIG. 10 is a flowchart of a signal processing method performed by the apparatus; and

FIG. 11 is a flowchart of an alternative signal processing method performed by the apparatus.

### DETAILED DESCRIPTION

### Overview

Enhanced physiological tremors and skin temperature changes such as caused by vasoconstriction, in a human body can be associated with, and can be a symptom of, hypoglycaemia. Hypoglycaemia may be referred to as a form of physiological event, or predetermined health condition: i.e., a hypoglycaemic health condition.

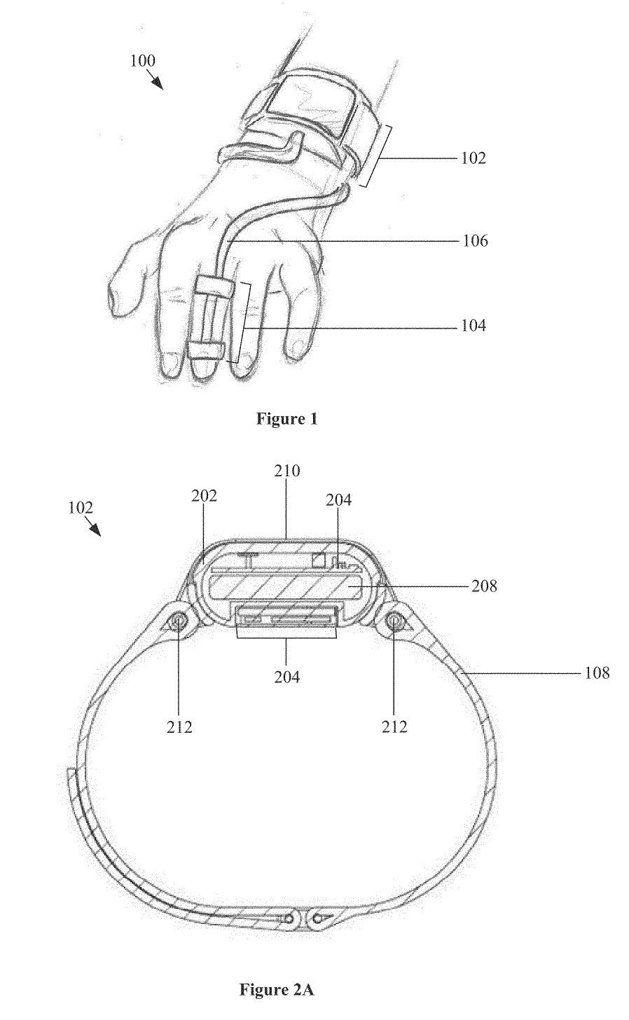

An apparatus 100, as shown in FIG. 1, is small and wearable by humans, including small children and large adults. The apparatus 100 can be referred to as a “wearable apparatus”, in the sense of “wearable computing”. The apparatus 100 includes a wrist assembly 102 and a finger assembly 104 connectable using a cable 106. The cable 106 may be regarded as an element of the finger assembly 104. The wrist assembly 102 is configured to be mounted to an arm, and can therefore be referred to as an “arm assembly”. In the description hereinafter, various components, measurements and steps are referred to in relation to the wrist as opposed to the finger; however, in embodiments, these various components, measurements and steps relating to the wrist can be placed and used in relation to the arm in general, in particular the forearm (between the wrist and the elbow), or the elbow and/or the upper arm, and the finger assembly 104 may be configured for use elsewhere on the hand, e.g., in the palm of the hand, or across the knuckles of the hand, and the sensors and predefined thresholds adjusted accordingly. As used herein, the term “finger”, without more specific reference to which finger, includes the thumb.

The apparatus 100 can warn people of an onset of hypoglycaemia by monitoring and measuring the physiological tremors and the skin temperature changes of a human wearing the apparatus. The tremors are associated with at least one oscillatory movement of the individual, including arm movements and finger movements. The human can be referred to as the wearer, or an individual, or a person. The apparatus 100 monitors movement (acceleration and rotation) at an arm or a wrist of the wearer using the wrist assembly 102 and monitors movement (acceleration) of an associated hand at an associated finger of the wearer using the finger assembly 104. The associated finger is one of the fingers on a hand associated with the wrist, e.g., the right-hand wrist and one of the fingers on the right hand, or the left-hand wrist and one of the fingers on the left hand. The associated finger may be one of the four lateral fingers, including one of the index finger, the middle finger, the ring finger and the little finger. The apparatus 100 monitors the human's skin temperature and ambient air temperature to provide indications of physiological effects that cause the skin temperature change, such as vasoconstriction, in the arm (wrist) and hand (finger). Signal processing methods 1000 and 1100, described hereinafter, include local signal processing steps performed locally by the apparatus 100 to determine or estimate whether the wearer is about to experience, or is experiencing, a hypoglycaemic event, by processing the received signals to generate an output (signal) indicative of hypoglycaemia in the wearer. The output (signal) that is indicative of hypoglycaemia is used to alert the wearer, or another person caring for the wearer. By storing and processing data based on signals from its sensors, the apparatus 100 provides continuous monitoring of the wearer (which can be periodic or spaced by intervals), even while the wearer is sleeping, thus allowing the wearer or a carer of the wearer (e.g., a friend, family member or health professional) to provide assistance (e.g., administering glucose) or seek medical help. In some embodiments, the apparatus 100 can be used to ameliorate risks from type-1 diabetes and night-time hypoglycaemic events, and—in some embodiments—type-2 diabetes. In some embodiments, the apparatus 100 may be used to monitor sleeping children with the aim of alerting the child, or a carer, if there is a risk of a potentially fatal hypoglycaemic event.

Described herein is an apparatus for a human to wear, the apparatus including an arm movement sensor and a finger movement sensor for generating concurrent movement measurements of an arm and of a corresponding finger of the human.

The apparatus can include any one or more of: an arm assembly with the arm movement sensor and an arm mount for mounting on the arm; a finger assembly with a finger movement sensor and a finger mount for mounting on the finger; an electronic system in electronic communication with the arm assembly and the finger assembly, wherein the electronic system is configured to receive the arm movement measurement from the arm movement sensor and the finger movement measurement from the finger movement sensor, and generate an alert signal representing a physiological event of the human based on the movement measurements; and at least one glove for the arm movement sensor and/or the finger movement sensor, including a single-finger glove. The arm can be a wrist.

The movement sensors include one or more linear movement sensors, and/or one or more angular movement sensors.

Described herein is an apparatus for a human to wear, the apparatus including a movement sensor and a temperature sensor for generating concurrent movement measurements and temperature measurements of the human.

The apparatus can include any one or more of:

an arm assembly with the movement sensor and/or the temperature sensor;

a finger assembly with the movement sensor and/or the temperature sensor;

an electronic system that compares the temperature measurements from the temperature sensor with a predetermined temperature reference or temperature difference to generate an alert signal representing a physiological event of the human.

Described herein is an apparatus for a human to wear, the apparatus including an arm temperature sensor and a finger temperature sensor for generating concurrent temperature measurements of an arm and of a corresponding finger of the human.

Described herein is an apparatus for a human to wear, the apparatus including two or more temperature sensors for generating concurrent temperature measurements from the human and from an ambient environment.

The apparatus can include one or more of:

an arm assembly with two of the temperature sensors; and

a finger assembly with two of the temperature sensors.

Described herein is an apparatus for a human to wear, the apparatus including one or more pressure sensors for generating a pressure measurement representing pressure between the apparatus and the human.

The apparatus can include one or more of:

an electronic system that determines whether the apparatus is worn properly, that determines whether the apparatus is constrained; and/or that generates an alert signal if the pressure measurement is outside predetermined acceptable measurements;

an arm assembly with one of the one or more pressure sensors;

a finger assembly with one of the one or more pressure sensors;

an electronic system that communicates with an external communicating device.

Described herein is an apparatus for a human to wear, the apparatus including an electronic system that controls at least one conductive connection to operate in two states including:

a first state in which the conductive connection receives direct current (DC) power for charging a battery and in which the conductive connection transmits and receives communication data according to a first protocol; and

a second state in which the conductive connection provides DC power from the battery and in which the conductive connection transmits and receives communication data according to a second protocol.

The apparatus can include an arm assembly with the conductive connection, and finger assembly with a cable that connects to the conductive connection in the second state.

Described herein is a method including steps of an arm movement sensor and a finger movement sensor generating concurrent movement measurements of an arm and of a corresponding finger of a human.

The method can include steps of an electronic system receiving the arm movement measurement from the arm movement sensor and the finger movement measurement from the finger movement sensor, and generating an alert signal representing a physiological event of the human based on the movement measurements.

Described herein is a method including steps of a movement sensor and a temperature sensor generating concurrent movement measurements and temperature measurements of an arm and/or a corresponding finger of a human.

The method can include steps of an electronic system comparing the temperature measurements from the temperature sensor with a predetermined temperature reference or temperature difference to generate an alert signal representing a physiological event of the human.

Described herein is a method including steps of an arm temperature sensor and a finger temperature sensor generating concurrent temperature measurements of an arm and of a corresponding finger of the human.

Described herein is a method including steps of two or more temperature sensors generating concurrent temperature measurements from the human and from an ambient environment.

Described herein is a method including steps of one or more pressure sensors in a wearable apparatus generating a pressure measurement representing pressure between the pressure sensors and a human.

The method can include the steps of an electronic system determining whether the apparatus is worn properly, determining whether the apparatus is constrained; and/or generating an alert signal if the pressure measurement is outside predetermined acceptable measurements.

Described herein is a method including the steps of:

a conductive connection, in a first state, receiving direct current (DC) power for charging a battery, and transmitting and receiving communication data according to a first protocol; and

the conductive connection, in a second state, providing DC power from the battery, and transmitting and receiving communication data according to a second protocol.

Described herein is a wearable apparatus for assessing at least one health parameter of an individual, including:

one or more sensors for measuring respective health parameters of the individual, said health parameters including at least one oscillatory movement of the individual; and

at least one data processing unit configured to:
receive signals representative of the measured health parameters; and
process the received signals to generate an alarm signal for alerting the individual to at least one predetermined health condition.

The sensors can include at least one of an accelerator and a gyroscope for measuring at least one oscillatory movement of the individual. The sensors can include a sensor for measuring at least one oscillatory movement of a finger of the individual. The sensors can include a sensor for measuring at least one oscillatory movement of an arm of the individual. The sensors can include a temperature sensor for measuring a temperature of the individual.

The wearable apparatus can further include a reference temperature sensor for measuring a temperature of an environment of the individual. The wearable apparatus can further include a pressure sensor for measuring a pressure of attachment between the wearable apparatus and the individual.

The at least one data processing unit can be configured to process a signal representative of the measured pressure of attachment to determine whether the wearable apparatus is properly mounted to the individual, and to generate an alarm signal for alerting the individual if the wearable apparatus is not properly mounted to the individual.

The wearable apparatus can include a display for displaying information and alerts to the individual.

The processor can be configured to process the received signal to generate an alarm signal for alerting the individual to a hypoglycaemic health condition.

The wearable apparatus can include at least one of an audio transducer and a vibration transducer to alert the individual to the at least one predetermined health condition.

The wearable apparatus can further include at least one wireless communications transceiver to allow the wearable apparatus to communicate with at least one external device or system.

Described herein is a method of using a wearable apparatus to assess at least one health parameter of an individual, including steps of:

using one or more sensors to measure respective health parameters of the individual, said health parameters including at least one oscillatory movement of the individual; and

using at least one data processing unit to:
receive signals representative of the measured health parameters; and
process the received signals to generate an alarm signal for alerting the individual to at least one predetermined health condition.

Described herein is an apparatus for generating an alert based on concurrent measurements from a wrist-mounted accelerometer and a finger-mounted accelerometer. The alert can be a hypoglycaemia alert.

Described herein is an apparatus for generating a hypoglycaemia alert based on concurrent measurements from one or more accelerometers and one or more temperature probes. The accelerometer can be one or more wrist accelerometers and/or a finger accelerometer. The temperature probe can be a wrist probe and/or a finger probe. The electronic system can compare a temperature measurement from the temperature probe with a predetermined temperature reference or temperature difference.

Described herein is an apparatus for generating a hypoglycaemia alert based on concurrent measurements from one or more accelerometers, and one or more gyroscopes. The accelerometer can be one or more wrist accelerometers and/or a finger accelerometer. The gyroscope can be a wrist gyroscope and/or a finger gyroscope.

Described herein is an apparatus for detection of temperature changes (e.g., due to vasoconstriction) by measurement of finger and wrist temperatures using a plurality of temperature sensors for skin and reference temperature from an environment.

Described herein is an apparatus for generating an alert if the apparatus is not fitted properly (e.g., because the apparatus is not worn or is incorrectly fitted/improperly mounted), or is constrained (because the apparatus is constrained or pinned, e.g., by the wearer's body) based on a pressure sensor. The pressure sensor may be a finger-mounted pressure sensor and/or a wrist-mounted pressure sensor.

The apparatus can generate an alert output, based on an alert signal, using a display, an audio speaker, a vibration motor, and/or an external communicating device that includes a wireless transceiver for communicating with the apparatus. The apparatus can be configured to send the alert signal to the external communicating device using a wireless communications protocol.

Described herein is an apparatus for generating an alert based on concurrent measurements from wrist-mounted sensors and finger-mounted sensors.

Described herein is an apparatus for indicating hypoglycaemia in a human, the apparatus including:

a wrist-mountable assembly with one or more wrist accelerometers and a wrist mount for wearing on a wrist of the human;

a finger-mountable assembly with a finger accelerometer and a finger mount for wearing on a finger of the human; and

an electronic system in electronic communication with the wrist-mountable assembly and the finger-mountable assembly, wherein the electronic system is configured to receive a wrist acceleration signal from one or more wrist accelerometers and a finger acceleration signal from the finger accelerometer, and generate an alert signal based on concurrent acceleration measurements from one or more wrist accelerometers and the finger accelerometer indicative of hypoglycaemia in the human.

Described herein is a method for generating a hypoglycaemia alert based on concurrent measurements from one or more accelerometers and one or more temperature probes.

Described herein is a method for generating a hypoglycaemia alert based on concurrent measurements from one or more accelerometers and one or more gyroscopes.

Described herein is a method for detection of temperature changes (e.g., due to vasoconstriction) by measurement of finger and wrist temperatures using two or more temperature sensors for determining skin and reference temperature of an environment.

Described herein is a method for generating an alert if the apparatus is not fitted properly based on a pressure sensor (e.g., because the device is not worn or not correctly fitted).

Described herein is a method for generating an alert based on concurrent measurements from wrist-mounted sensors and finger-mounted sensors.

Described herein is a method for indicating hypoglycaemia in a human, the method including steps of:

a wrist-mountable assembly generating a wrist acceleration signal;

a finger-mountable assembly generating a finger acceleration signal;

an electronic system receiving the wrist acceleration signal from the wrist-mountable assembly;

the electronic system receiving the finger acceleration signal from the finger-mountable assembly; and

the electronic system generating an alert signal based on concurrent acceleration measurements from the wrist acceleration signal and the finger acceleration signal.

### Mechanical System

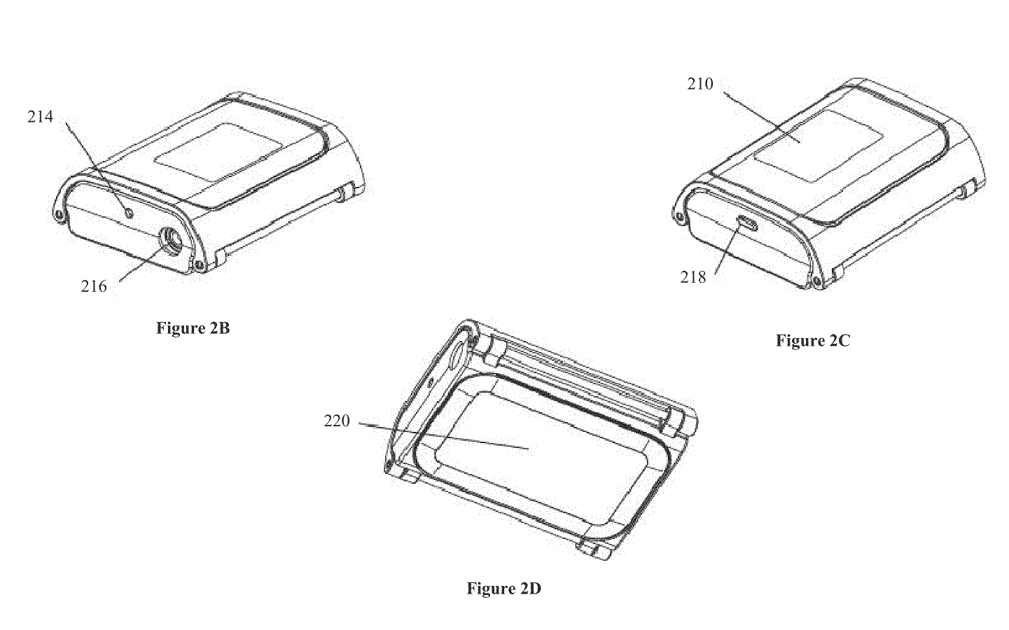

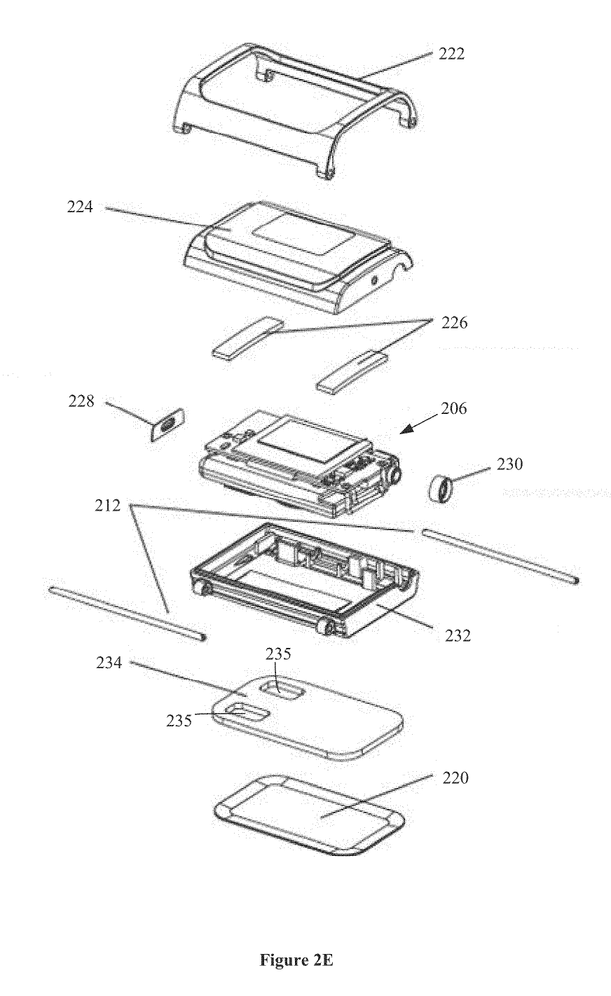

As shown in FIG. 2A, the wrist assembly 102 includes a wrist band 108, which is a band or strap used to secure the wrist assembly 102 to the wrist. The band 108 can be formed of neoprene fabric.

As shown in FIGS. 2A, 2B, 2C, 2D and 2E, the wrist assembly 102 includes:
a wrist housing 202 around the outer part of the wrist assembly 102 for protecting the other components of the wrist assembly 102 from ingress of dust and moisture, and from mechanical impacts, and for holding the other components in place;
wrist electronics 204 (which is an electronic system) inside the wrist housing 202, including a printed circuit board assembly (PCBA) 206, described hereinafter, and a battery 208;
a wrist display cavity 210 on an outer side of the wrist assembly 102 for mounting a display 302 displaying visual information under control of the wrist electronics 204;
a wrist-band coupler, which can include wrist band pins 212, that attaches the wrist band 108 to the wrist assembly 102;
an audio aperture 214 (which can be referred to as a buzzer port) through which sound from an audio speaker 316 inside the housing 202 can pass; the audio aperture 124 can include an air-permeable water-sealed membrane, adhered to an inner side of the audio aperture 214, to improve ingress protection of the audio aperture 214;
a multifunctional connector aperture 216 for a multifunctional connector 328 described hereinafter;
a reset control aperture 218 for a reset control 322 (which can be a button) described hereinafter;
a back plate 220 on an inner side of the wrist assembly 102 for contacting the skin of the wearer, and arranged to conduct a force from the skin to the wrist electronics 204, and to conduct heat from the skin to the wrist electronics 204;
a metal facia 222 attached to the outer side of the wrist assembly 102 for attachment of the wrist band 108 to upper and lower housings 224 and 232 via wrist band pins 212;
an upper housing 224 of the housing 202, located and attached towards the outer side of the wrist assembly 102;
upper thermally conductive compliant elements 226, which can be foam, adhered and located between the wrist electronics 204 and the upper housing 224, that seal the sides of the display 302 to reduce light scatter from the display 302;
a reset seal 228 (which can be a button boot) to improve ingress protection around the reset control 322;
a multifunctional connector seal 230 to improve ingress protection around and through the multifunctional connector 328;
a lower housing 232 of the housing 202, located towards the inner side of the wrist assembly 102; and
a lower thermally conductive compliant element 234 located and adhered between the back plate 220 and the lower housing 232.

The lower thermally conductive compliant element 234 conducts heat from the skin and arm to a skin temperature sensor 310 described hereinafter. The element 234 is compressed by the lower housing 232 and back plate 220 to seal the inner side of the wrist assembly 102 to provide some ingress protection against moisture and dirt. The lower thermally conductive compliant element 234 includes two holes 235 that align to a skin temperature sensor 310 and a skin accelerometer 312, described hereinafter, to provide space for these components.

The wrist band 108 and the wrist housing 202 forms a wrist mount to mount the wrist assembly 102 to the arm of the wearer, which can be the wrist portion or end of the arm. The wrist assembly 102 can alternatively or additionally include at least one glove that fits around the arm or the wrist to mount the wrist assembly 102 to the arm of the wearer, which can be the wrist portion or end of the arm.

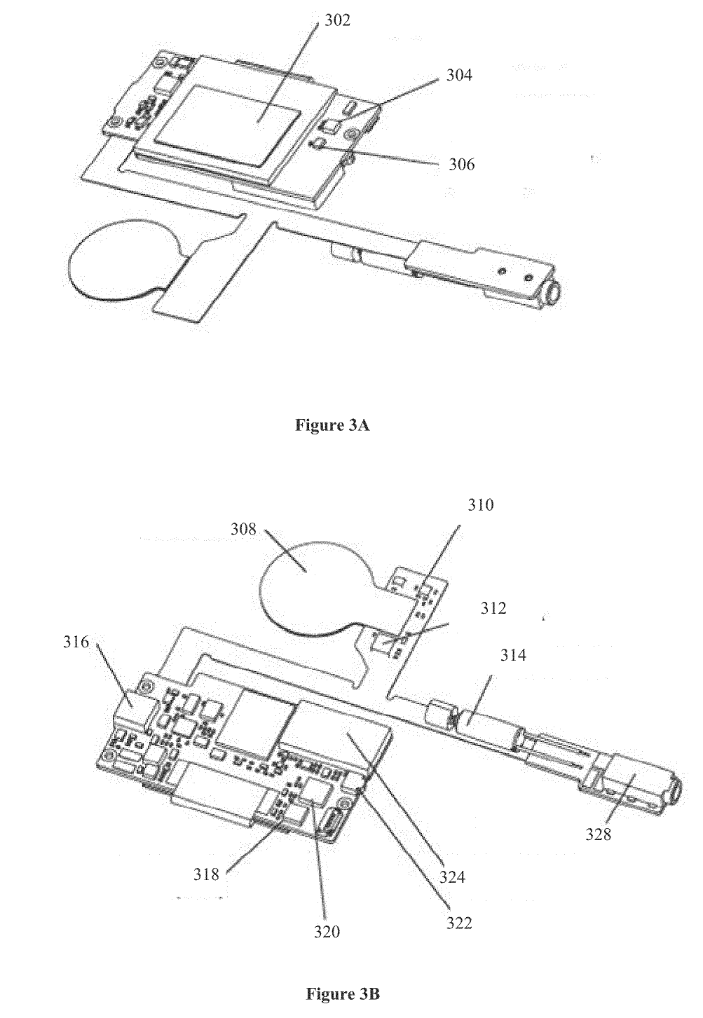

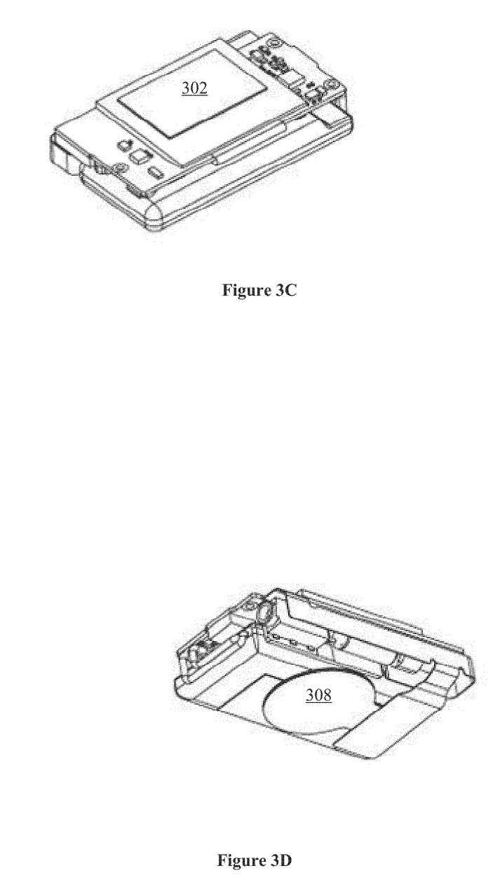

As shown in FIGS. 3A and 3B, the wrist electronics 204 include:
the display 302, which can be an organic light-emitting diode (OLED) display, which—as shown in FIG. 3C—is at the outer side of the electronics 204 when the PCBA 206 is assembled;
an ambient light sensor 304 that measures an ambient light level in order to change the intensity of the display based on the ambient light level;
a reference temperature sensor 306 that is located towards the outer side of the wrist assembly 102 for measuring a reference temperature (i.e., an ambient air temperature) from the close ambient environment around the wearer (e.g., under bed-clothes or at wearer-height within the room), in contrast to a skin temperature of the wearer;
a pressure sensor 308 that is located towards the inner side of the wrist assembly 102 to measure the pressure between the skin and the wrist assembly 102; as shown in FIG. 3D, when assembled, the pressure sensor 308 is located at the innermost side of the wrist electronics 204 and adjacent, and adhered to, the compliant element 234 to measure pressure between the housing 202 (which is applied by the wrist band 108 via the wrist-band coupler) and the skin of the wearer; the pressure sensor 308 generates a pressure signal that is used by the apparatus 100 to determine appropriate fitment of the wrist assembly 102 to the wearer, as described hereinafter;
the skin temperature sensor 310 that is located towards the inner side of the wrist assembly 102 for measuring the arm skin temperature, in contrast to the ambient temperature of the room or environment: the skin temperature sensor 310 is located on a region of the PCBA 206 adjacent the lower thermally conductive compliant element 234 to receive heat flow from the skin, through the back plate 220 (which is thermally conductive) and through the element 234 (which is thermally conductive) and through the PCBA 207 (which is thermally conductive);
an inner wrist accelerometer 312 that is also located towards the inner side of the wrist assembly 102 for measuring acceleration close to the skin: the wrist accelerometer 312 is located on a region of the PCBA 206 adjacent the lower compliant element 234 to receive accelerations (tremors or vibrations within predetermined frequencies) from the arm on which the wrist assembly 102 is attached, through the back plate 220 (which conducts vibrations) and through the element 234 (which conducts vibrations) and through the PCBA 207 (which conducts vibrations);
a vibration motor 314 and the audio speaker 316 (which can be a buzzer), both of which are electrically connected to the battery 208 and a microcontroller 508 of the apparatus 100 for activation when the microcontroller 508 generates an alert;
an outer wrist accelerometer 318 that is also located towards the outer side of the wrist assembly 102 for measuring acceleration more directly coupled to the wrist via the wristband than is the case for the inner wrist accelerometer 312 (in some embodiments, only one of these accelerometers 318,312 is included and used);
a gyroscope 320 (also referred to as a “gyroscopic sensor”) that generates and sends angular acceleration signals to the microcontroller 508 for use in a signal processing method 1000 described hereinafter;
the reset control 322 that can be manually actuated by a person, which can include covering or pressing by a finger, to send a reset command to a reset controller 522, described hereinafter;
a wireless communications module 324 (which can be a Bluetooth module operating according to the Bluetooth™ protocols) that is in electronic communication with the microcontroller 508, and is configured to operate according to at least one wireless communications protocol, and thus allows wireless communication between the microcontroller 508 and an external communicating device 718 described hereinafter; and
the multifunctional connector 328 (which may be referred to as a “finger sensor connector port” or an external sensor connector or a “smart connector” or a charging port), the functionality of which is described hereinafter.

In embodiments, the reference temperature and the skin temperature will both be influenced by the ambient environmental temperature, and the body/skin temperature of the wearer; however, the reference temperature is based more closely on the ambient environmental temperature than the skin/body temperature, and—conversely—the skin temperature is based more closely on the skin/body temperature than the ambient environmental temperature.

The external communicating device 718 includes a wireless transceiver for communicating with the apparatus 100. The apparatus 100 is configured to send alert signals to the external communicating device 718 to form a personal area network (PAN) with the apparatus 100 and the external communicating device 718. The apparatus 100 and the external communicating device 718 can communicate using the at least one wireless communications protocol, which can be a low-energy protocol, which can include an ANT protocol, an ANT+ protocol, a ZigBee protocol, a Bluetooth (BT) protocol, a cellular communications protocol, and/or a WiFi protocol. The external communicating device 718 may be referred to as a “separate wireless device”, and can include an iPhone (from Apple Inc.), an Android phone (from Samsung or other manufacturer), an iPad with WiFi and/or cellular connectivity, a Windows phone, an iPod Touch, a Personal Computer (PC), a wireless router, a docking station with WiFi and/or broadband connectivity, and/or a smart watch. A PC may be a desktop computer or a laptop, netbook, tablet or a handheld PC (or palmtop).

The vibration motor 314 has a small footprint, with a diameter of 8 mm and a height of 3.4 mm. Due to a high current draw of the motor 314, it is powered directly from the battery 208, rather than through the voltage regulators 512.

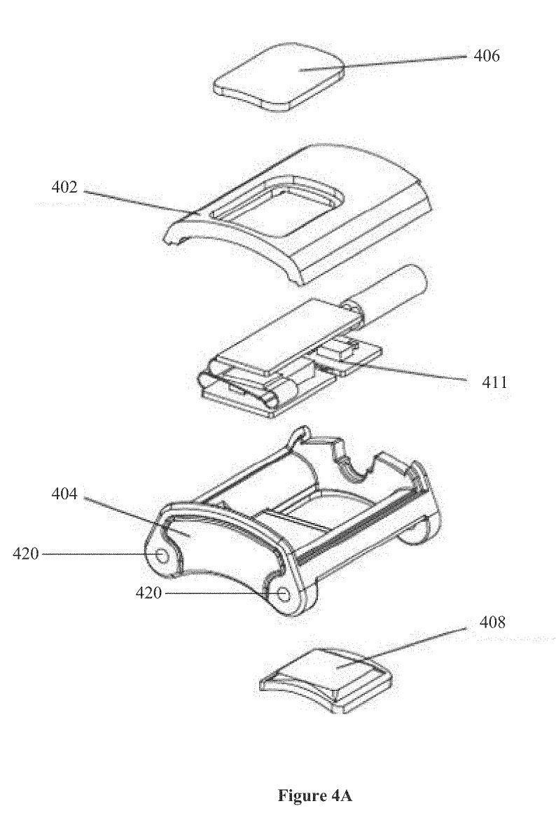

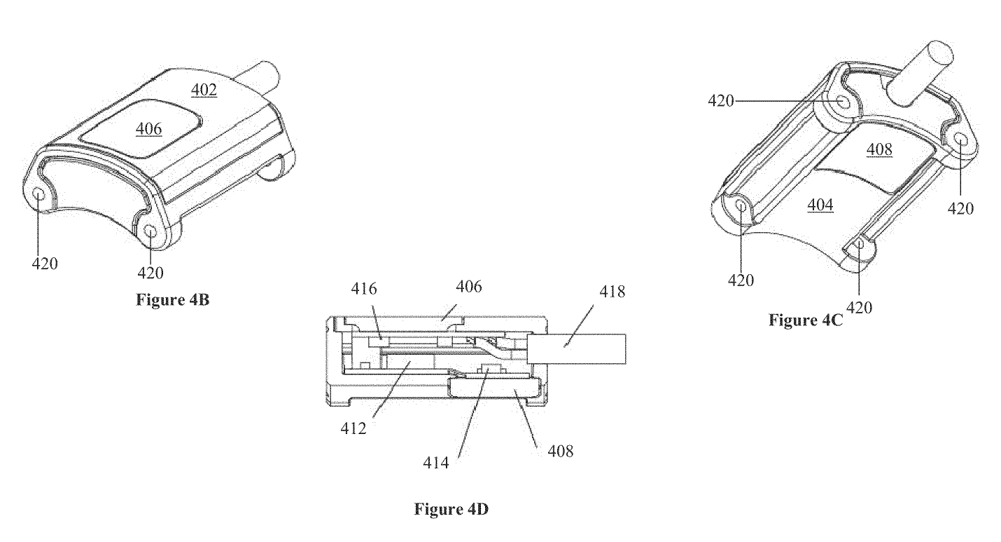

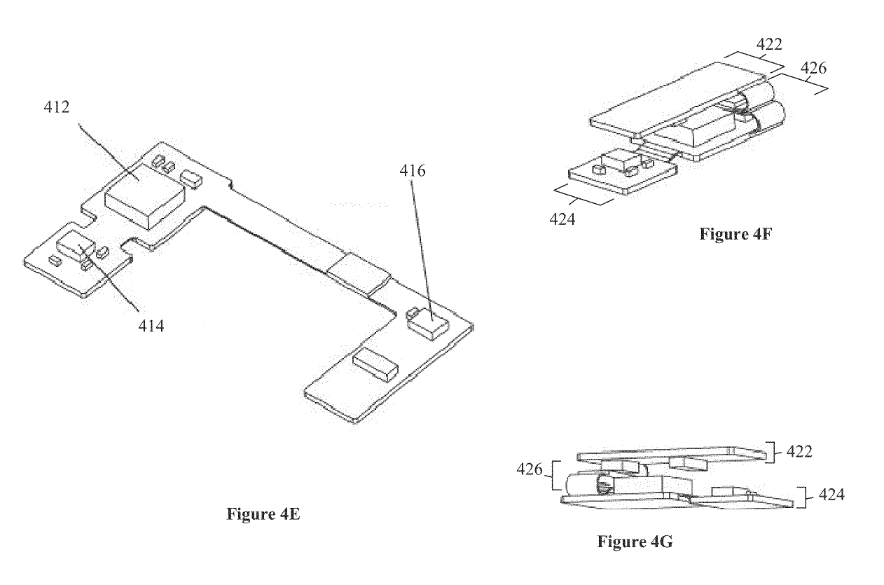

As shown in FIG. 4A, the finger assembly 104 includes:
an upper housing 402 on the outer side of the finger assembly 104;
a lower housing 404 on the inner side of the finger assembly 104 that attaches to the upper housing 402 to provide a finger housing to protect and locate the other components of the finger assembly 104;
a top plate 406, located in an aperture of the upper housing 402, that is thermally conductive to conduct heat from the surrounding room or environment to a reference temperature sensor 416 described hereinafter;
a back plate 408, located in an aperture of the lower housing 404, that is thermally conductive to conduct heat from the finger skin of the wearer to a skin temperature sensor 414 described hereinafter;
finger electronics (i.e., electronic components forming an electronic system), inside the finger housing, including a finger printed circuit board assembly (PCBA) 411; and
holes 420 to hold retention pins connectable to a least one band, which can be a band or strap, which can be made of neoprene including a band fastener, which together form a finger mount to mount the finger assembly 104 to the finger of the wearer.

In embodiments, the finger assembly 104 can include at least one glove, which can be a single-finger glove, for mounting (or securing or attached) the finger assembly 104 to the finger. The finger glove can form a portion of the glove for the wrist assembly 102 described hereinbefore.

In some embodiments, the finger assembly 104 can include a finger pressure sensor that is similar to the pressure sensor 308 described hereinbefore. The finger pressure sensor generates finger pressure measurement signals based on the pressure of the finger assembly 104 on the finger, and these are transmitted to the microcontroller 508 for processing in a similar series of steps to the processing of the wrist pressure data, including generation of finger-pressure alert signals.

As shown in FIGS. 4D, 4E, 4F and 4G, the finger electronics include:
a finger accelerometer 412 that measures acceleration of the finger to which the finger assembly 104 is attached and sends a corresponding time-domain finger acceleration signal to the microcontroller 508;
a finger skin temperature sensor 414 that is located towards an inner side of the finger assembly 104 for measuring the finger skin temperature, in contrast to the reference temperature of the room or environment: the finger skin temperature sensor 414 is located on a region of the finger PCBA 411 adjacent the thermally conductive back plate 408 to receive heat flow from the skin, through the back plate 408 (which is thermally conductive) and through the finger PCBA 411 (which is thermally conductive);
a finger reference temperature sensor 416 that is located towards the outer side of the finger assembly 104 for measuring a reference temperature around the finger, in contrast to the finger skin temperature; and
a wire harness 418 for the cable 106.

The accelerometers 312,318,412 and the gyroscopic sensor 320 are movement sensors (also known as “motion sensors”) that sense movement of the human. The movement sensors and the temperature sensors 310,306,414,416 may be referred to as “health parameter sensors” that sense or measure health parameters of the human, or “physical property sensors” that sense or measure respective physical properties (i.e., movement and temperature) of the human.

The sensors, including the temperature sensors 310,306,414,416, the movement sensors (the accelerometers 312,318,412 and the gyroscopic sensor 320), and the pressure sensor 308, generate and send respective electronic signals to the microcontroller 508 for use in the signal processing method 1000. The electronic signals represent measurements made by the respective sensors, and can be referred to as “measurement signals”. Each of the movement sensors can generate and send three independent measurement signals representing respective measurements in three spatial directions, e.g., along X, Y and Z orthogonal axes for acceleration, and roll, pitch and yaw for angular acceleration. The measurement signals can represent absolute or relative measurements of the physical values (acceleration, angular acceleration, temperature), or can be indicative of these value, e.g., generating discrete values like a switch. Each measurement signal includes a plurality of the measurements over time that together form a waveform or time series of the measurement values. The measurement signals can be analogue signals that are converted to digital signals, and then to data series by the microcontroller 508, or the sensors can generate digital signals and/or data sequences representing the time-domain measurements directly. The sensors operate “concurrently” in the sense that they operate at the same time, and make measurements during the same time period or periods: the measurements from the different sensors may be strictly simultaneous, but may also be at slightly different times within acceptable time differences, depending on the speed of the sensors, the rate of change of the properties being measured, and the sampling and processing speeds of the microcontroller 508: sampling rates are described hereinafter, and the “concurrent” measurements can be considered to be the measurements made within each sampling period. The different sensors can have different sampling rates.

In the wrist assembly 102, and in the finger assembly 104, the temperature sensors are located on the PCBAs in such a way as to provide thermal isolation between skin sensors and reference sensors. The wrist reference temperature sensor 306 is at a different lateral position in the assembled PCBA 206 from the wrist skin temperature sensor 310. Similarly, the finger reference temperature sensor 416 is at a different lateral position in the assembled PCBA 411 from the finger skin temperature sensor 414. The PCBAs 206, 411 use copper pads to locally improve the thermal conductivity of the PCBAs 206, 411 and thin copper tracks to increase the thermal isolation of one temperature sensor 206, 414 from another 306, 416, respectively. The PCBAs 206, 411 are flexible in portions, and they are assembled by bending or folding a first sub-region 422 over a second sub-region 424, including a plurality of folds or bends in a third sub-region 426 between the first sub-region and the second sub-region, e.g., as shown in FIGS. 3C, 3D, 4F, 4G. This allows the temperature sensors on each PCBA to be separated by a longer distance on the PCBA than the direct distance between the temperature sensors in each assembly, 102,104, thus improving the thermal isolation of the skin and reference temperature measurements. In an alternate embodiment, the PCBAs 206, 411 may be comprised of multiple sections joined by connectors, for cost-effectiveness and ease of assembly.

### Electronic Systems

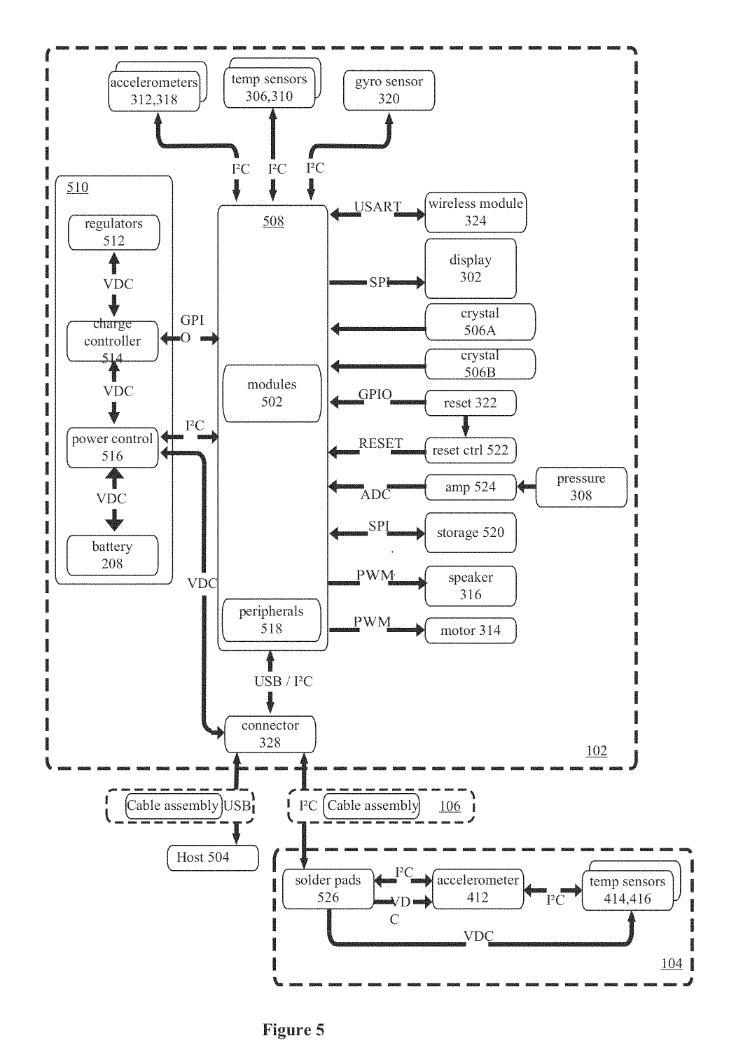

As shown in FIG. 5, the acceleration sensors 312,318 or accelerometers transduce movement into electrical acceleration signals that are sent to the microcontroller 508 using an I2C bus. The accelerometers can be packaged as devices. In embodiments, the accelerometers can be mono-axial, bi-axial or tri-axial acceleration sensor devices. Each accelerometer package can have a size of 3×3×1 mm. The accelerometers can be low-power accelerometers with selectable accelerometer sensitivity of ±2, 4 and 8 Earth's gravities full-scale ranges (which can be pre-selected in configuration data). The accelerometers can have 14 bits of resolution, providing a resolution of 0.00025 Earth's gravities. The sampling rate of the accelerometer can be in the range 1.56 to 800 samples per second. The accelerometers can provide I2C output directly to the microcontroller 508, and the I2C address of each accelerometer is selected with a hardware pin, thus more than one device can share one I2C bus. As shown in FIG. 5, the apparatus 100 includes at least two I2C buses: one bus internal to the wrist assembly 102, and another bus that is switched through the multifunctional connector 328, thus allowing at least the possible 3 accelerometers 312,318,412.

The sampling rate of the gyroscope can be in the range 100 to 800 samples per second. The Gyroscope Sensitivity can be in the range+/−250 to +/−1000 degrees per second. The Gyroscope Resolution can be 16 bit. The gyroscopic sensor 320 can be packaged as a device. In embodiments, the gyroscope can be a single axis device or multiple-axis gyroscope device with two or three different rotational axes that are used to generate the measurements. The gyroscopic sensor 320 can provide I2C output directly to the microcontroller 508. In embodiments, in order to conserve battery charge, the gyroscope can be briefly disabled (e.g., for 5 seconds) when signal processing outputs (particularly those using lower-power sensors, such as arm position-determination in step 1010) indicate that the conditions briefly do not allow observation of physiological phenomena of interest.

The apparatus 100 includes computer-readable non-volatile data storage 520, which may include flash memory, that stores (or “records”) sampled data generated by the apparatus 100 during monitoring. The sensor data can be stored in its raw form, or can be compressed in a lossless, summary or selective fashion (which can include storing data during suspected hypoglycaemic events only). The signal processing output can also be stored to the flash memory.

The apparatus 100 includes a rechargeable battery 208, which can be a high-density lithium-polymer rechargeable battery with inbuilt safety features (including a power control module 516 that disconnects the battery if any fault is detected). The battery can have a minimum capacity of 260 mA hour (nominally 280 mA hour), and can be 4.5 mm×30 mm×26 mm in size without leads. The apparatus 100 includes a charge controller 514 dedicated for the battery 208 that controls a charge profile for battery charging, and a power circuit 510 (also referred to as a “battery charger”) including regulation and protection circuits that convert battery charge into stable power for the other electronic components in the apparatus 100. The apparatus 100 includes a voltage regulator 512, with low-dropout linear characteristics, to regulate voltage to all components in the apparatus 100.

The apparatus 100 includes the display 302 to display information for the wearer. The display 302 may include indicator lights, which can be light-emitting diodes (LEDs), used to display user notifications. The indicator lights can include two indicator lights to display four alerts or states. The user notifications may include the alerts, described hereinafter. The user notification may include an operational notification, or an Operational State, showing constant green, when the apparatus 100 is receiving signals and logging or recording data.

The apparatus 100 includes a Tap Sensor that allows the apparatus 100 to receive user input from the wearer to acknowledge reminders. The Tap Sensor may be provided by means of one of the movement sensors described herein.

The apparatus 100 includes the temperature sensors 306,310 that provide temperature signals or data representing temperature measurements of the arm skin and finger skin of the wearer, and of ambient reference temperatures, as described hereinbefore.

The apparatus 100 includes at least one microcontroller 508 (which can be a microcontroller or microcontroller unit, “MCU”, or one or more data processing units) with circuits and embedded modules 502 that provide the local signal processing steps for the apparatus 100 to perform. Some embodiments can include a plurality of microcontrollers in communication with each other in the apparatus 100. Some embodiments can include one or more microcontrollers that are configured to perform at least portions of the functions of the microcontroller 508 in an external housing from the microcontroller 508 described herein, and a communications protocol can be used to unity the functions of the microcontrollers. The microcontroller 508 can be an ultra-low power microcontroller based on a 32-bit ARM Cortex-M3 RISC processor, housed in a small, 7×7 mm package, configured for portable low power applications. The microcontroller 508 has on-chip peripherals 518, that support unpowered serial data exchange protocols (which can include Inter-Integrated Circuit (I2C) protocols, Serial Peripheral Interface (SPI) protocols, RS-232 protocols, and/or RS-485 protocols), and powered data protocols (which can include a Universal Serial Bus (USB) protocol, and/or a Power over Ethernet (PoE) protocol). As shown in FIG. 5, the serial data exchange protocols can include an SPI interface for direct connection to the storage 520 and the display 302, and I2C ports to communicate with the sensors. The powered data protocols can include a USB interface to communicate with host computer 504, which can be a personal computer (PC), a laptop computer or a tablet computer, having a commercially available operating system (e.g., Windows or MacOS). The host computer 504 can be referred to as a “host device”, or an “external computer”.

The microcontroller 508 has an on-chip real time clock (RTC), requiring one or more external frequency-setting crystals 506A, 506B. The RTC is used to time-stamp the recorded data. Firmware on the microcontroller 508 can be changed or updated from the host computer 504. The RTC can keep track of information since the apparatus was last configured with the host computer 504. The RTC can have a resolution of 1 second, can be accurate to within 60 seconds per day, and can be synchronized to a mobile device, including the external communicating device 718.

The wireless communications module 324 can communicate wirelessly with the external communicating device 718. The wireless module 324 includes a wireless transceiver that supports one or more of the protocols of the external communicating device 718 described hereinafter, which can be a Bluetooth (BT) low-energy transceiver. The wireless module 324 may have a range of up to 10 metres. The wireless module 324 is in electronic communication with the microcontroller 508 using a Universal Synchronous/Asynchronous Receiver/Transmitter (USART) protocol. The wireless module 324 can monitor the wireless connection with the external communicating device 718, and can generate alert signals for the microcontroller 508 if the wireless connection is interrupted or lost, which can include generating an alert indicative of a loss of Bluetooth pairing.

The pressure sensor 308, which can include a force-sensing resistor, generates an analogue signal that is sent to an amplifier 524 that, in turn, send the pressure measurement signal to an analogue-to-digital converter input of the microcontroller 508.

The finger assembly 104 includes solder pads 526 that connect the cable 106 to the DC power and unpowered communication protocol connections of the finger sensors 412,414,416.

### Multifunctional Connector

The apparatus 100 includes the multifunctional connector 328 that provides two functions in two respective modes or states:

(1) in a charging mode (which can be referred to as a charging/communications mode), the multifunctional connector 328 connects the microcontroller 508 and the power circuit 510 (according to one of the powered data protocols described hereinbefore) to the host computer 504 for data upload, data download, and battery charging; and

(2) in a monitoring mode (which can be referred to as a data bus mode), connecting the interface of one of the unpowered serial data exchange protocols (described hereinbefore) to the finger assembly 104 while the apparatus 100 is in its Monitor State.

The multifunctional connector 328 may be referred to as “smart” because it has three distinct modes of operation. The multifunctional connector 328 can be a port or jack or socket. Alternatively, the multifunctional connector 328 can be a plug. The finger assembly 104 may be referred to as an “external sensor” because it is external to the wrist assembly 102.

The multifunctional connector 328 provides at least one conductive connection for the electronic communications and the power connection between the apparatus 100 and the host computer 504 for configuration and connection of the apparatus 100, for electrical charging, and for data download. An external charger can charge the battery by connecting to the multifunctional connector 328.

The multifunctional connector 328 also provides the same at least one conductive connection for the electronic communications and power connection between the wrist assembly 102 and the finger assembly 104. The at least one conductive connection can be single conductor, or single conductive element having a single voltage and current node.

The apparatus 100 includes a data switch, which can be configured to switch between protocols (for the two respective functions mentioned hereinbefore), that is both the externally powered and unpowered protocols.

The apparatus 100 can include a connector cover to improve ingress protection of the multifunctional connector 328.

The multifunctional connector 328 can be implemented using an audio jack, which can be a four-pole 2.5-mm tip, ring, ring, sleeve (TRRS) jack. The jack, although commercially available, can be selected to be an uncommon size, e.g., 2.5-mm instead of the more common 3.5-mm version. This multifunctional connector 328 supports 4 poles suitable for either the powered or unpowered protocol connection to the host computer 504 or the finger assembly 104 respectively. The multifunctional connector 328 incorporates a mechanical switch. Alternatively, a 5-pole connector can be used, with the additional pole tied to ground within the finger assembly 104, providing an alternate switch mechanism. When a mating connector that supports the powered protocol or the unpowered protocol (e.g., a plug from a USB device, a charger, or an I2C device) is connected to the multifunctional connector 328, the switch allows the microcontroller 508 to detect the presence of the connected mating connector.

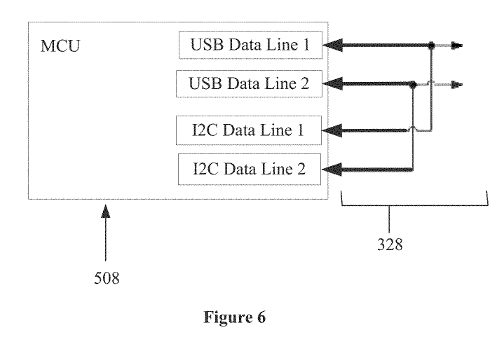

As shown in FIG. 6, the multifunctional connector 328 provides physical data bus line connections linked in parallel to the microcontroller 508 for switching between the powered protocol and the unpowered protocol.

To control the multifunctional connector 328, the microcontroller 508 performs a switching method 900.

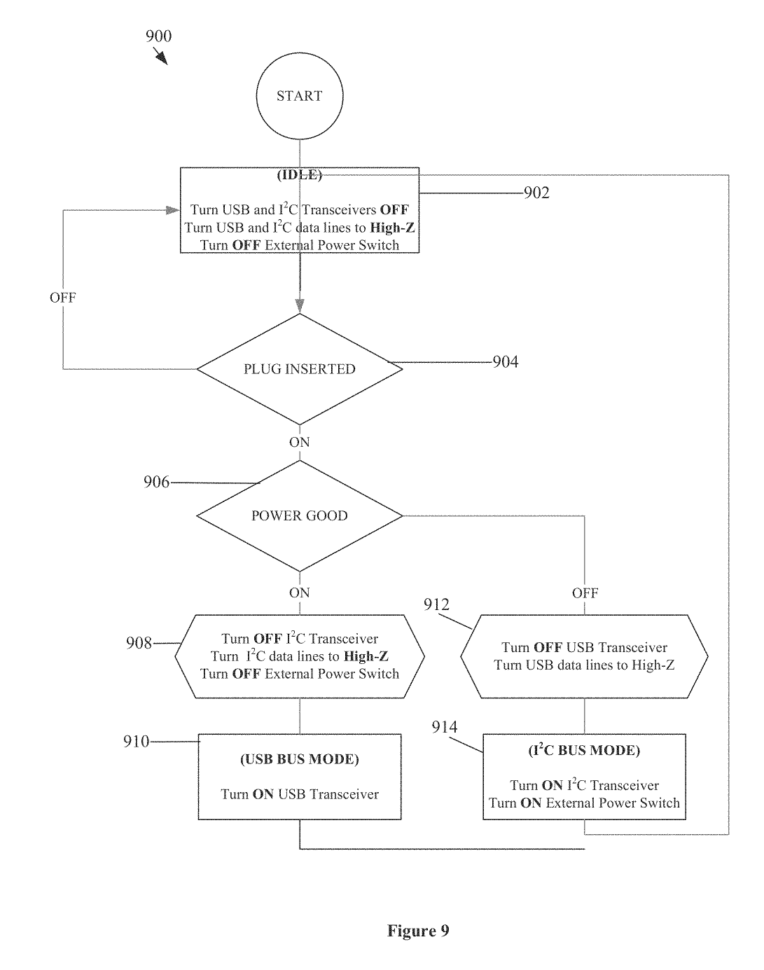

As shown in FIG. 9, and in relation to an embodiments switching a USB powered protocol and an I2C unpowered protocol, the switching method 900 includes steps of:
starting in an Idle State, with the mating connector disconnected, or when the mating connector is removed, both the powered protocol and the unpowered protocol transceivers are turned OFF, and the data bus signals of the microcontroller 508 (USB D+, USB D−, I2C SCL and I2C SDA) in a high impedance (High-Z) state (step 902);
the microcontroller 508 receives a signal when the mating connector is connected (which can include a plug being inserted into the jack) via the “plug inserted” GPIO input (step 904);
the microcontroller 508 determines or identifies a connection type of the mating connector by measuring a connector input voltage provided by the mating connector: if a charging voltage is detected (which can be +5 VDC from a USB host or a USB charging device), providing a “power good” indication to the microcontroller 508 by a second GPIO input signal that is indicative of the powered protocol (step 906);
if the “power good” input is “ON”, as determined in step 906, the microcontroller 508 turns “OFF” the unpowered protocol transceiver (if it was previously enabled), puts the data line pins (SDA and SCL) into a high impedance (High-Z) state, and turns the powered protocol transceiver “ON” (step 908);
after turning “ON” the powered protocol slave transceiver, this slave transceiver pulls up one of the powered protocol data lines (USB D+ or D−) to indicate the presence of a connected powered protocol host, and the powered protocol slave transceiver commences data transfer and battery charging once the connected host is identified (step 910);
if the “power good” input is “OFF”, as determined in step 906, the microcontroller 508 turns “OFF” the powered protocol transceiver (if it was previously enabled) and puts its data line pins (USB D+ and D−) into a high impedance (High-Z) state, turns the unpowered protocol transceiver along with an external power switch “ON”, and the external power switch also connects DC power from the battery 208 to the finger assembly 104, via the regulators 512, to power it: the regulated DC battery power is configured to be at a different and lower voltage (+3.0-3.3 VDC, set by the voltage regulators 512) than the powered source voltage (step 912); and
after the unpowered protocol transceiver is turned “ON” and connected to power, the unpowered protocol transceiver commences unpowered protocol data communication (step 914).

The I2C bus specification requires pull-up resistors on each data line to the bus line voltage. These pull-up resistors are included only in the finger assembly 104 because inclusion of these pull-up resistors in the wrist assembly 102 would incorrectly identify the wrist assembly 102 while in powered protocol mode to a USB host device.

In step 904, plug insertion may be determined from the mechanical switch. Alternatively in this step, the switch can be omitted or ignored if the data line pins (SDA and SCL) are set as inputs with weak pull-downs enabled (rather than High-Z), and a logic high on these signals is used to indicate the presence of the finger assembly 104, wherein the finger 104 has stronger pull-ups than in the pull-downs applied by the microcontroller 508 for these pins and signals.

In step 906, the external power signal triggers the unpowered protocol transceiver to switch off after the jack detection step 904; alternatively, step 906 can be performed before step 904, or can always take precedence.

In step 914, the multifunctional connector 328 is connected to the data power from the battery 208 (which can be the +3.3 VDC source), so the charge controller is configured to differentiate between the data power source (a +3.3 VDC level) and the charging voltage of the powered protocol (+5 VDC) so that the “power good” signal is not set back to “ON” during the monitoring mode.

### Reset Controller

The apparatus 100 includes an electronic reset controller 522 that provides a state-dependent reset mechanism. If the apparatus 100 stops working as expected, e.g., due to software, firmware or hardware failure, the reset mechanism restarts the apparatus 100 and the apparatus 100 subsequently re-commences normal operation (including the switching method 900 and the Signal Processing Method 1000).

The reset controller 522 has two inputs. If both inputs are held for more than 7.5 seconds, reset controller 522 executes a hardware reset of the microcontroller 508. One input is connected to a user input button. The other input is connected to the charge controller's “power good” output. Thus, the hardware reset can only be invoked by: first connecting the apparatus 100 to a valid charging source; and second holding the user input button for more than 7.5 seconds. In this way, accidental resets are prevented or reduced while the apparatus 100 is in normal use. This can be important if the wearer is a child, or is asleep.

### Alerts

An alert can include an alert signal, indicative of an alert condition being met in the method 1000. An alert can also include an alert output that is observable or detectable by a person, such as the carer or the wearer, by one of his or her five physical senses. An alert can be referred to as an “alarm” or a “warning” or a “notification”.

The apparatus 100 can generate an alert output, based on an alert signal, using the display 302, the audio speaker 316, the vibration motor 314, and/or the external communicating device 718. Thus, in operation, the apparatus 100 can display the alert, generate an audible alert, and/or generate a vibration alert.

The alerts can include:
a hypoglycaemia alert (which can be referred to as a warning alert) indicative of suspected hypoglycaemia based on the alert signal (this can control the display 302 to flash red, and the audio speaker 316 and the vibration motor 314 to activate);
a low-battery alert, or a low-battery state (which can include flashing or constant amber), when the battery charge or voltage drops to or below a preselected threshold, which can represent 10% of a full battery charge, as determined by the power circuit 510;
a connection-loss alert based on the alert signals generated by the wireless module 324 if the wireless connection to the external communicating device 718 is interrupted or lost, as described hereinbefore;
a poor fitment alert when the pressure measurements are outside the predetermined acceptable values, as determined by the microcontroller 508;
a constrained alert, or a pinned alert, when the pressure measurements are outside the predetermined acceptable values, as determined by the microcontroller 508;
a fault alert, constant or flashing amber, when a data-processing fault or electronic fault is detected by the microcontroller 508; and
an interference alert when interference signals (including due to excessive vibration, such as what may occur when used on an overnight train, or due to temperature extremes), which might interfere with hypoglycaemia detection by the microcontroller 508, are detected above a pre-selected level during monitoring mode, where the signal processing method 1000 or 1100 is active.

The different alerts can be differentiated by various means including the urgency of escalation and by the information displayed on the display 302 and/or the external communicating device 718, and by different activation sequences of the audio speaker 316 and the vibration motor 314.

### Software Model

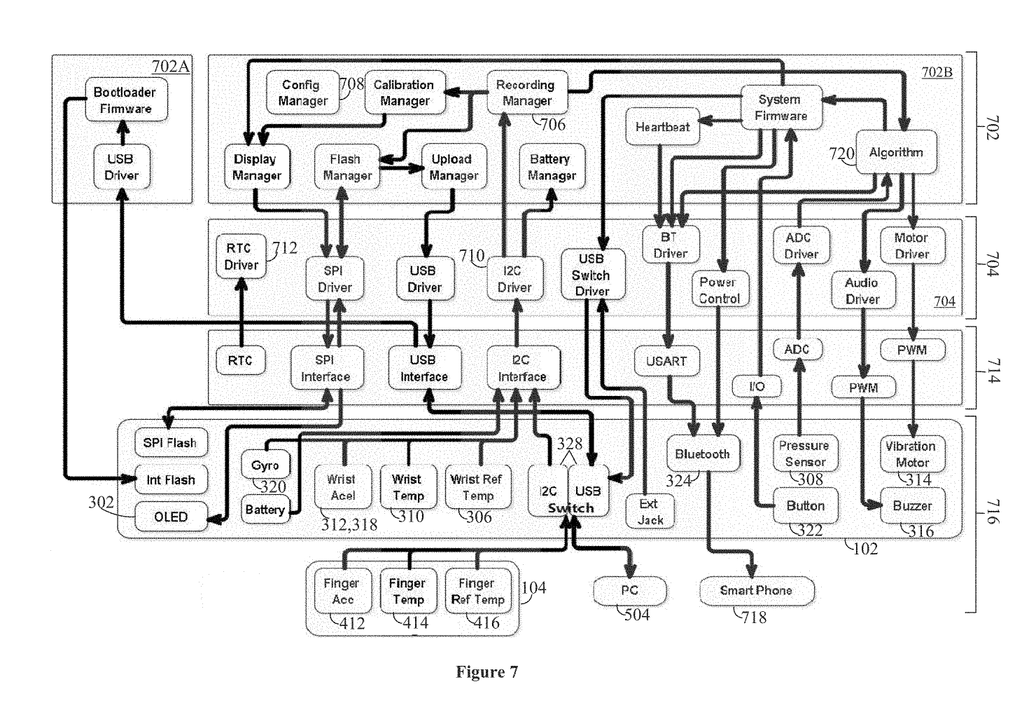

As shown in FIG. 7, the apparatus 100 includes an application layer 702 that is responsible for controlling and coordinating activity of the apparatus 100, including tasks such as initializing drivers, processing events and subsequently controlling other managers and peripherals. The application layer 702 includes a bootloader 702A and an application 702B.

The application 702B includes a Recording Manager 706 that provides an interface for recording the accelerometer, gyroscope, pressure sensor and temperature sensor data, the processed data (from the signal processing method 1000) and the debugging data into the storage 520. A recording rate from the signal processing method 1000 can be in the range 0-800 samples/second.

The application 702B includes a Configuration Manager 708 (“Config Manager”) for recording the configuration data from the host computer 504 in the storage, extracting the configuration data from the storage on power up, and making the configuration data available to the microcontroller 508 during operation.

In some embodiments, the application 702B can include a File System Driver that provides a file system interface to the storage. The File System Driver can perform cluster allocation during initial start-up. The configuration data, recording data and debug data are stored in separate files in the file system interface, which allows for retrieval of the stored data directly when the apparatus 100 is connected to the host computer 504 as a Mass Storage Device (MSD). A Serial Flash Driver can be used to control the flash memory, including controlling data transfer and issuing initialization commands to the flash memory prior to the start of recording, such as erasure of offloaded information and write-enabling. In alternative embodiments, the configuration data may effectively be included in the compiled code of the apparatus 100, and there is no need for a separate File System Driver.

The apparatus includes a driver layer 704 with drivers for the apparatus 100. The driver layer 704 includes an I2C Driver 710 for configuring sensor peripherals based on data provided from the Config Manager 708, and for passing sensor data readings available for the Recording Manager 706.

The driver layer 704 includes a real-time clock (RTC) Driver 712 that generates accurate date/time information from a periodic timer interrupt and dictates the overall timing resolution.

All device drivers in the driver layer 704 are event-driven and non-blocking. The application layer 702 is also event-driven and non-blocking. Some blocking is allowed for initialization code that is sequential by nature.

The driver layer 704 collects data from the temperature sensors. Temperature is recorded at 1 sample per second.

The driver layer 704 includes a general-purpose input-output (GPIO) driver to provide an interface for GPIO based peripherals and the apparatus 100. The GPIO driver passes a USB Detect Event when the host computer 504 is connected to the apparatus 100. The GPIO driver, when commanded by the application layer 702, enables or disables the multifunctional connector 328. The GPIO driver, when commanded by the application layer 702, turns on or off the user-notification indicators such as vibration motor, beeper and display.

The components of the application layer 702 and the driver layer 704 operate in the microcontroller 508.

As shown in FIG. 7, the apparatus 100 includes a processor support layer 714 with electronic components in communication with the drivers.

The apparatus 100 includes a hardware layer 716 with the wrist assembly 102, the finger assembly 104, the host computer 504 and the external communicating device 718.

### State Machine Modes

The microcontroller 508 operates according to a plurality of interconnected operational states or modes 800 provided by a state machine of the apparatus 100.

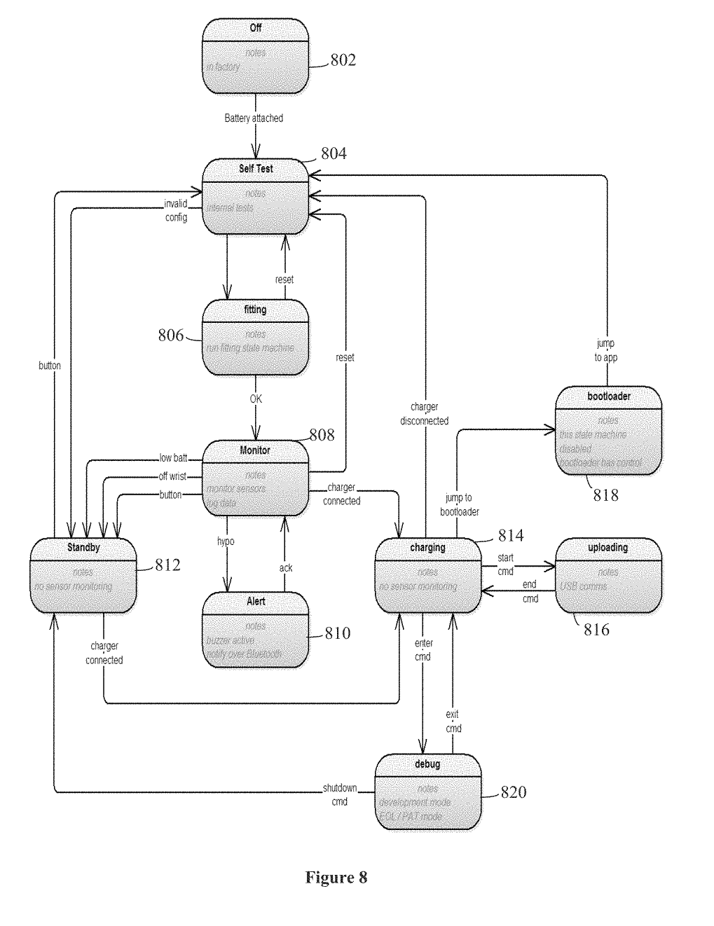

As shown in FIG. 8, the modes 800 include an Off Mode 802, in which the apparatus 100 has no power, and a Self-Test Mode 804, which is reached from the Off Mode 802, during which the microcontroller 508 executes internal testing routines.

The modes 800 include a Fitting Mode 806, reached from the Self-Test Mode 804, during which the apparatus 100 is fitted to the finger and arm, and during which the pressure sensor 308 can provide feedback signals or alerts for the wearer to fit at least the wrist assembly 102 to within predefined acceptable pressures; the Self-Test Mode 804 can be reached from the Fitting Mode by control of the reset control 322 (although only if the power is attached to the multifunctional connector 328, as described hereinbefore).

The modes 800 include the monitoring mode in the form of a Monitor Mode 808 (which may be referred to as a Normal Usage Mode), reached from the Fitting Mode, 806 in which the apparatus 100 operates continuously until the battery level is too low, and in which the apparatus 100 measures and processes the Acceleration Data and Wrist Sensor Data (but does not record them), and in which the Acceleration Data is analysed by the signal processing method 1000 for error correction and tremor detection, and when a tremor is detected, the Visible Reminder and Vibrating Reminder are activated. The Self-Test Mode 804 can be reached from the Monitor Mode 808 by control of the reset control 322 (although only if the power is attached to the multifunctional connector 328, as described hereinbefore).

The modes 800 include an Alerting Mode 810, reached from the Monitor Mode 808 when an event is detected (which can include a hypoglycaemic event), in which alert signals and data are generated for the display 302, the vibration motor 314, the audio speaker 316, and the BT module 324. The microcontroller 508 can reach the Monitor Mode 808 from the Alert Mode 810 if an alert is acknowledged or cancelled, which can be by appropriate activation of the reset control 322.

The modes 800 include a Standby Mode 812 (which may be referred to as a Battery Too Low Mode), which is entered from the Monitor State 808 when apparatus 100 detects that the battery charge/voltage is dropping below a pre-configured Battery Too Low Threshold, and in which the apparatus 100 shuts down completely after writing system state information into the storage, thus attempting to preserve battery chemistry and to minimise battery damage due to over discharge, and in which the display 302 is turned off, and in which no power is drawn from the battery 208. The microcontroller 508 can also enter the Standby Mode 812 from the Monitor Mode 808 if the pressure sensor 308 measures zero pressure, and the microcontroller 508 determines that the wrist assembly 102 has been removed from the wearer.

The modes 800 include the charging mode in the form of a Charging Mode 814, in which the apparatus 100 is in a low power mode, the accelerometers are off, data logging and the signal processing method 1000 are disabled, and the battery level is read periodically (which can be every 5 minutes). The microcontroller 508 can enter the Charging Mode 814 from the Monitor Mode 808 if the charger is connected to the multifunctional connector 328. The microcontroller 508 enters the Self-Test Mode 804 if the charger is disconnected in the Charging Mode 814. The microcontroller 508 enters the Charging Mode 814 from the Standby Mode 812 if the charger is connected.

The modes 800 include an Uploading Mode 816 (also known as a Configuration and Data Extraction Mode), which is entered when the apparatus 100 is connected to the host computer 504, and in which data logging and the signal processing method 1000 are disabled, to configure parameters of the signal (i.e., during the training, or threshold determination, of the apparatus 100) processing method 1000, and to retrieve data logged during Manual Learning Mode. The microcontroller 508 enters the Uploading Mode 816 from the Charging Mode 814 when an appropriate command is received from the host computer 504 and/or the external communicating device 718. The microcontroller 508 exits the Uploading Mode 816 to the Charging Mode 814 when an end command is received from the host computer 504 and/or the external communicating device 718.

The modes 800 include a Bootloader Mode 818 in which the state machine is disabled and in which the firmware of the microcontroller 508 can be updated. The Bootloader Mode 818 is entered from the Charging Mode 814, and exited to the Self-Test Mode 804. The Bootloader Mode 818 runs in the apparatus 100 and can be used to update or re-install the modules 502 of the other modes, e.g., to effect a firmware update.

The modes 800 include a Debugging Mode 820 for development, testing and debugging of the microcontroller 508 and the other electronic components in the apparatus 100. The microcontroller 508 enters the Debugging Mode 820 from the Charging Mode 814 when an appropriate command is received from the host computer 504 and/or the external communicating device 718. The microcontroller 508 exits the Debugging Mode 820 to the Charging Mode 814 or to the Standby Mode 812, if an appropriate command is received from the host computer 504 and/or the external communicating device 718.

### Signal Processing Methods

In embodiments, the microcontroller 508 is configured to perform a signal processing method 1000 in which signals from the sensors are processed for storage, and for activating an alert if necessary. The signal processing method 1000 is performed continuously by the apparatus 100 when in the Monitor Mode 808.

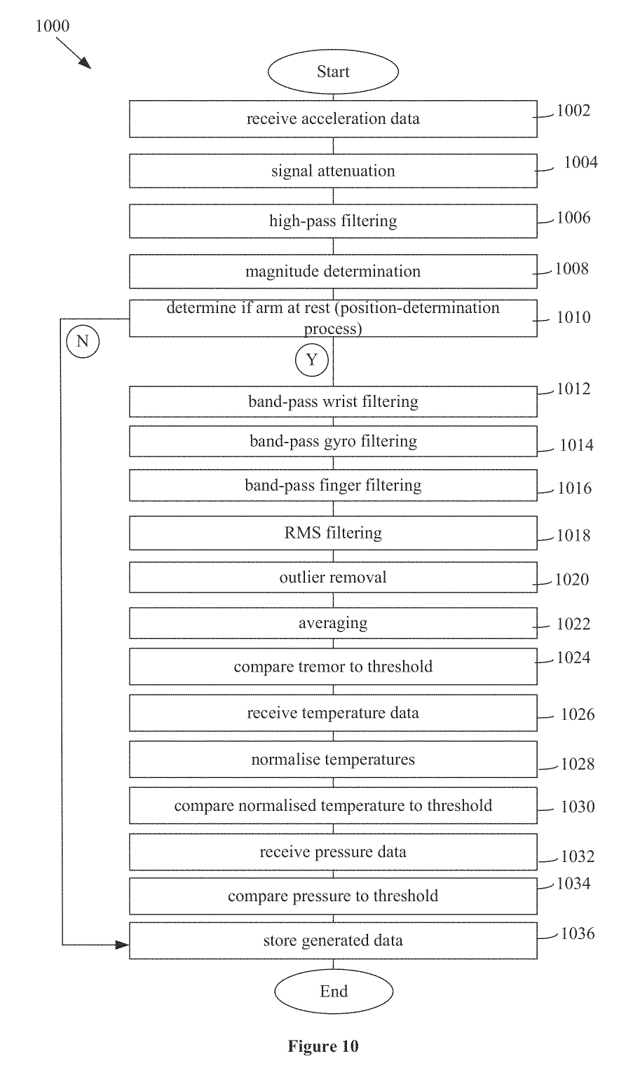

As shown in FIG. 10, in the signal processing method 1000:
the microcontroller 508 receives sampled data representing the acceleration signals (representing acceleration in three dimensions) from the accelerometers and gyroscopic signals (representing roll, pitch and yaw) from the gyroscope (step 1002);
the microcontroller 508 executes a progressive signal attenuation process, in which the microcontroller 508 compresses the received acceleration signals to have magnitudes within a preselected operating range in order to limit the influence of large movements on subsequent processing stages: for example, the received acceleration signals may have a received minimum magnitude of 0.0 Earth's gravities (e.g., in a direction perpendicular to gravity) a received maximum magnitude of 3.46 Earth's gravities (e.g., acceleration from movement of the sensor plus gravity), and the received values can be mapped or compressed, e.g., linearly, to fall between a minimum of 0.0 Earth's gravities and a maximum of 1.3 Earth's gravities (step 1004);
the microcontroller 508 applies high-pass digital filters to each received acceleration signal, including a first high-pass filter to remove a rolling mean of a preselected duration (e.g., over 2 seconds), and a second high-pass filter (which may be referred to as a “pre-filter”) to remove the mean (e.g., due to gravity), and to reduce low-frequency noise (e.g., offsets due to slow changes in temperature, voltage, etc.), based on a preselected low cut-off frequency (e.g., 4 Hz) (step 1006);
the microcontroller 508 determines a vector magnitude for each orthogonal group of accelerometer signals, X, Y and Z, e.g., determined according to the relationship: (vector magnitude)=square-root of (X²+Y²+Z²) (step 1008);
the microcontroller 508 does not process the acceleration values unless the wearer's arm is determined to be at rest when the values were measured in a position-determination process described hereinafter, accordingly, the microcontroller 508 determines if the arm is at rest: if the arm is not at rest, the method 1000 merely progresses to recording the data in step 1036, and finishes; if the arm is determined to be at rest, the microcontroller 508 proceeds to the filtering steps 1012, 1014, 106 (step 1010);
the microcontroller 508 band-pass filters the acceleration signals to remove frequency components that are not indicative of the medical condition of interest (hypoglycaemia) based on preconfigured frequencies of interest, e.g., known from clinical studies on cohorts of humans, or on previous monitoring of the individual wearer, or through continuous adaptation of filters. For example, to detect hypoglycaemia, the wrist acceleration signals (including linear and/or angular acceleration) can be filtered between 8 Hz and 12 Hz (e.g., using a filter that has −6 dB at 7.9 Hz, −3 dB at 8 Hz, −3 dB at 12 Hz, −6 dB at 12.1 Hz, and a logarithmic decay); and the finger acceleration signals can be filtered between 18 and 30 Hz (e.g., using a filter that has −6 dB at 17.9 Hz, −3 dB at 18 Hz, −3 dB at 30 Hz, −6 dB at 30.1 Hz, and a logarithmic decay) (steps 1012, 1014, 1016);
the microcontroller 508 applies a root-mean-squared (RMS) filter to determine RMS mean values over measurement intervals of preconfigured averaging durations, e.g., over 100 sample using a 100-tap RMS filter (step 1018);
the microcontroller 508 removes outlier values within each measurement interval by removing any values greater than a mean-absolute deviation from the RMS mean for the corresponding measurement interval (step 1020);
the microcontroller 508 generates an average vector magnitude value for each measurement interval (step 1022);
the microcontroller 508 compares the generated average vector magnitude value to one or more preconfigured and continuously adapted threshold values or ranges for each acceleration sensor or combination thereof, and generates an output signal indicative of hypoglycaemia if the generated value is outside the threshold values or range, which may be adjusted based on other sensor inputs such as temperature, and actives an alerting process based on the output signal (step 1024);
the microcontroller 508 receives temperature data representing the temperature signals from the temperature sensors 306,310,414,416, including the skin temperatures and the ambient temperatures on the wrist assembly 102 and the finger assembly 104 (step 1026);
the microcontroller 508 normalises the skin temperatures based on the respective reference temperatures by subtracting, or otherwise cancelling, the reference temperature at each measurement time from the skin temperatures at each measurement time (step 1028) in order to represent the differential between skin and reference temperatures, and the subsequent differential between finger and wrist;
the microcontroller 508 compares the normalised temperature at each measurement time (which extends over a preconfigured temperature-measurement duration) to at least one preconfigured and continuously adapted threshold values, and generates an output signal indicative of hypoglycaemia if the normalised value is outside the threshold values or range, which may be adjusted based on other sensor inputs such as accelerometer, or if there is a deviation in the normalised value from a previously established steady-state value, e.g., there is a significant drop in finger and/or wrist temperature due to vasoconstriction, and activates the alerting process based on the output signal (step 1030);
the microcontroller 508 receives pressure data representing the pressure signals from the pressure sensor 308, including the skin pressure between the wearer's skin and the wrist assembly 102, and/or between the wearer's skin and the finger assembly 104 (step 1032);
the microcontroller 508 compares the pressure at each measurement time (which extends over a preconfigured pressure-measurement duration) to at least one preconfigured pressure threshold or range, and actives the alerting process if the measured pressure is outside one or more preconfigured values or ranges, which indicated that the apparatus 100 is not being worn, or is not fitted correctly, or is constrained or pinned by a weight, e.g., the wearer's body because he or she has rolled onto the apparatus 100 during sleep), or if there is a deviation in the measured pressure from a previously established steady-state pressure, e.g., there is significant sustained drop or increase in on-wrist pressure (step 1034); and
the microcontroller 508 records (or stores) the generated data from the microcontroller 508, and the measurements from the sensors: the microcontroller 508 can be configured to record all sensor data, and record all outputs of the signal processing, regardless of whether the alert determination process is commenced from step 1012 (step 1036).

The pressure sensing and pressure alerting steps 1032,1034 can be used to provide active feedback while the apparatus 100 is being fitted to a wearer: the apparatus 100 may continue to provide alerts of a selected type until the apparatus 100 is fitted to have a preselected pressure on the pressure sensor 308, or to be within a preselected acceptable pressure rage, and once an acceptable pressure is measured, the alerts can stop and/or a “correct” alert can be generated (e.g., a “success” sound or symbol on the display 302).

The preconfigured thresholds or ranges (which may be defined as or relative to baseline values) are selected in a pre-configuration process (which may be referred to as a training process) that includes monitoring a wearer by the apparatus 100 used in the Monitor Mode 808 and which may have the alerting functionality inhibited for the duration of this process, and concurrently by at least one independent sensor. During monitoring, these preconfigured thresholds or ranges are continuously adapted based on activity and independent sensors. The independent sensor can be one or more blood-glucose (BGL) sensors. The blood-glucose sensors can include a Continuous Glucose Monitoring (CGM) sensor, which can be inserted into body tissue, e.g., abdominal fat, and provides a continuous estimate of blood glucose. The blood-glucose sensors can include a finger-prick sensor used more than once each day, e.g., seven times per day, to give accurate BGL measurements. The measurements of the physiological parameter from the independent sensors are recorded over a relatively long training period (e.g., a plurality of days or weeks or a month), during which the condition of interest, which can be hypoglycaemia, occurs once or more often. The independent recorded measurements are made concurrently with the acceleration, gyroscopic, pressure and temperature measurements in the apparatus 100. The resulting time-series recordings are compared after measurement, e.g., by an analyst or clinician, to preconfigure the alerting thresholds or ranges for using in the signal processing method 1000.

In some embodiments, rather than simply commencing the alerting process when one of the measured accelerations or normalised temperatures crosses a preconfigured threshold, or moves into or out of one or more preselected ranges, the microcontroller 508 can perform a multivariate analysis and classification process based on the measured acceleration and temperature values and training data from the pre-configuration process and continuous monitoring thereafter.

The microcontroller 508 performs the position-determination process to determine whether the wearer's arm is at rest. The position-determination process includes steps of:
determine the vector magnitude, described hereinbefore;
determine that the arm is not at rest if the vector magnitude is greater than a preconfigured gross-movement threshold, which can be 0.1 Earth's gravities, at any time within a preconfigured position-determination duration, which can be 5 seconds, thus generally exclude walking, running, and gross arm movements;
determine that the arm is not at rest if average pressure sensor measurements over the position-determination duration are above a preconfigured too-tight threshold (which can be referred to as a “constrained” or “pinned” threshold), which can be configured during an individual fitting procedure with each wearer (the too-tight threshold can be selected to be 20% above a comfortably-tight pressure), thus generally exclude the arm being held, e.g., by the head or body resting on the arm; and
determine that the arm is not at rest if average pressure sensor measurements over the position-determination duration are below a preconfigured too-loose threshold, which can be configured during an individual fitting procedure with each wearer (the too-loose threshold can be selected to be 20% below the comfortably-tight pressure), thus generally exclude measurements when the apparatus 100 is poorly fitted on the arm.

The position-determination process can also use the temperature measurements from the temperature sensors to determine whether the respective wrist assembly 102 and/or the finger assembly 104 are being worn. If the skin temperature measurement is not close to typical skin temperature, when normalised by the reference temperature, then the apparatus 100 may have been removed, except if movement is detected in which case this may be indicative of severe vasoconstriction.

The microcontroller 508 may use the pressure data to normalise the acceleration values, e.g., because a device subjected to external compressive forces will not properly convert physiological movements into measurable accelerations.

In embodiments, the microcontroller 508 is configured to perform an alternative or additional signal processing method 1100 in which signals from the physical-property sensors are processed for storage, and for activating an alert if necessary. The signal processing method 1100 is performed continuously by the apparatus 100 when in the Monitor Mode 808.

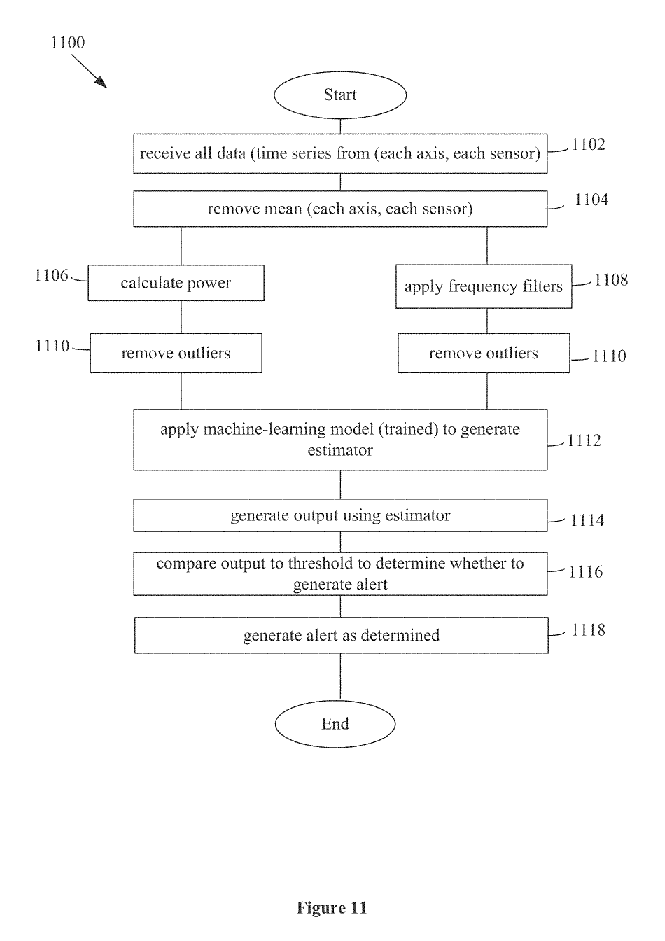

As shown in FIG. 11, in the signal processing method 1100:
the microcontroller 508 receives the sample data representing the measurement signals from the sensors, including the acceleration signals (representing acceleration in three dimensions) from the accelerometers and the gyroscopic signals (representing roll, pitch and yaw) from the gyroscope, i.e., with a separate time series of measurements for each sensor, and for each orthogonal axis of each movement sensor (thus each signal includes a plurality of values, e.g., 1024, with respective times during the measurement period): i.e., the movement sensors 312, 318, 412, 320 and the temperature sensors 310, 306, 414, 416, generate the time series of values for a predetermined measurement interval representing temperature values from the temperature sensors and three-dimensional (3D) measurements from the movement sensors: i.e., measurements along three orthogonal axes from the accelerometers, 312, 318, 412 and around three orthogonal axes from the gyroscopic sensor 320 (step 1102);
the microcontroller 508 removes the average or mean value (which may be referred to as a “DC value”) from each time series (i.e., the signals on each of the orthogonal axes of the movement sensors are treated as separate signals), including by determining the absolute value of the difference between each sample measurement instance (i.e., the measured value at a certain time “i”) and the mean value of that signal measured over the predetermined measurement interval (step 1104);
after removing the mean from each signal in step 1104, the microcontroller 508 determines a motion power or temperature power for each signal, including by integrating the absolute values of the plurality of values taken during the predetermined measurement interval, to give a “power” value, i.e., the sum of the squared values for the measurement interval, thus generating one power value for each signal (step 1106);
after removing the mean from each signal in step 1104, in parallel with step 1106, the microcontroller 508 also applies a frequency transformation, which can be a Fast Fourier Transform (FFT), to each signal from the movement sensors to compare the frequency components of each signal, and then the microcontroller 508 filters each signal by selecting two frequency components from each signal, in particular a first frequency component that is the strongest (e.g., has the highest absolute value) in a first frequency range (between the frequencies of 8 Hz and 12 Hz) and a second frequency component in a second frequency range (between the frequencies of 11 Hz and 15 Hz), thus selecting two of the strongest frequency-domain values for each signal from the movement sensors, e.g., thus providing 6 values for each 3D sensor (step 1108);
the microcontroller 508 removes any outliers from the power signal values from step 1106 and from the frequency component values from step 1108 based on a pre-determined maximum threshold, which can be the variance of the RMS value: if an outlier value is removed in this step, the removed value is replaced with a previous corresponding value from the same signal in a previous measurement interval (step 1110);
the microcontroller 508 applies the remaining values from steps 1106 and 1108, after removal of the outliers in step 1110, to a previously-trained machine-learning model (represented by data stored in the apparatus 100, e.g., in the computer-readable memory), i.e., by providing the remaining values as inputs to the machine-learning model: in embodiments, there can be one power-value input for each temperature sensor, and one power-value input and two frequency component inputs for each axis of each movement sensor (step 1112);
the microcontroller 508 uses the machine-learning model to determine an output (which can be a binary “yes” or “no” value), based on an estimator (which is a value) generated by the machine-learning model, with a pre-determined level of confidence for which the machine-learning model has been configured (step 1114);
the microcontroller 508 compares the output from the machine-learning model to a pre-determined value, which may be pre-configured for each individual wearing the device, and from this comparison generates an output signal indicative of hypoglycaemia: the output signal can be a binary “yes” or “no” value (step 1116); and
the microcontroller 508 activates the alerting process if the generated output corresponds to a pre-determined value representing hypoglycaemia for the human wearing the apparatus 100 and, in some embodiments, for whom the machine-learning model has been trained (step 1118).

The predetermined measurement interval for the concurrent measurement of the physical signals from the physical sensors can be between 1 second and 10 seconds, including 2 seconds, 3 seconds, 4 seconds, 5 seconds, 6 seconds, 7 seconds, 8 seconds, 9 seconds or 10 seconds. The predetermined measurement interval may be referred to as a “measurement period”. The data used in the signal processing method 1100 represent measurements from the sensors made during each measurement interval, and the measurements are therefore referred to as being concurrent measurements or contemporaneous measurements.

The machine-learning model is preconfigured using the selected confidence threshold (which can be 90%, or 95% or 99%) in the pre-configuration process described hereinbefore, in which the signals from the physical sensors are processed as described in the process 1100, and the machine-learning model is trained in a machine-learning process by providing the machine-learning system with the processed values as training inputs (i.e., corresponding to the “remaining values”) and indications of hypoglycaemia measured using at least one independent sensor during monitoring as a training target. The machine-learning model can be an artificial neural network with three layers, two layers, or one layer, and can include an Elliot symmetric sigmoid transfer function for each layer. The machine-learning model may be implementable in an artificial neural network (such as a non-linear clustering algorithm or a binary decision tree). The machine-learning model includes one or more layers, and a plurality of weights. The weights are values associating the input values to the output value, and the training process is used to determine the weights in the machine-learning model that provide the pre-selected confidence threshold for hypoglycaemia. The machine-learning process requires sufficient repetition for configuration of a reliable machine-learning model, which may be 500 nights of data across a broad range of diabetic individuals in one embodiment, or 7 nights of data on each individual wearer in an alternate embodiment. In the latter case, to keep the machine-learning model accurate for each individual, the training process can be repeated whenever sufficient time has elapsed such that a significant physiological change may have occurred, for example yearly. Significant physiological changes could include such phenomena as weight gain, weight loss, increased counter-regulatory hormone response to hypoglycaemia due to a reduced rate of hypoglycaemic events, puberty, or growth. In all cases, a number of further nights at least equal to the number of training nights is required to verify the effectiveness of the training, recorded under the same conditions.

In step 1114, the microcontroller 508 can generate the output to be equal to, or to otherwise directly correspond to, the estimator. Alternatively, the microcontroller 508 can generate the output using a deterministic adjustment of the estimator, e.g., a weighted combination of the current estimator with previous estimators, an integral including a plurality of previous estimators, a differential value between the current estimator and a previous estimator, or a difference between the current estimator and an average of previous estimators over a pre-selected estimator averaging duration, which may be 1 hour.

In step 114, the inputs are the results of signal processing of the sensors of the apparatus 100. Alternatively, additional inputs can be supplied which relate to the characteristics of the wearer of the apparatus 100, including age, height, weight, elapsed time since diagnosis of diabetes, gender, Gold hypoglycaemia awareness score and/or insulin sensitivity factor. Alternatively, additional inputs can be supplied which relate to the outputs of other signal processing steps, such as the arm position-determination process of step 1010, which provides an indicator of the wearer's degree of awakeness for use by the machine-learning model.

The microcontroller 508 includes compiled machine-readable code in computer-readable storage of the microcontroller 508. The compiled code defines instructions for the microcontroller 508 to perform the steps of the signal processing methods 1000, 1100. The compiled code is compiled from instructions written in the C or Matlab programming languages. The compiled code defines the algorithm 720 of the application layer 702, as shown in FIG. 7, that causes the apparatus 100 to operate according to the compiled code. The microcontroller 508 may include a single microcontroller unit or a plurality of units that are operatively connected and operate to provide the signal processing methods 1000, 1100. The microcontroller 508 normally processes information according to a program in the compiled code, i.e., a list of internally stored instructions, including calls to an operating system. The microcontroller 508 executes the compiled code to generate current program values and state information, and communicates with the other connected components of the apparatus 100. In some embodiments, the instructions may be embodied in the structure of circuitry that implements such functionality, e.g., firmware programmed into programmable or erasable/programmable devices, the configuration of a field-programmable gate array (FPGA), the design of a gate array or full-custom application-specific integrated circuit (ASIC), or the like. The data generation, data storage and data communications operations are digital data operations. The digital data includes electronic data defined by logic circuits—including binary logic circuits—generally represented by electronic quantities, including voltage, current and/or resistance.

In the signal processing methods 1000, 1100 the microcontroller 508 transmits a portion of, or all of, the data received in steps 1002, 1102 and generated subsequently, including the output generated values from the steps 1036, 1114, and the estimators from the machine-learning model, and transmits data representing these values to the external communicating device 718 for external storage and processing.

### Interpretation

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Many modifications will be apparent to those skilled in the art without departing from the scope of the present invention as hereinbefore described with reference to the accompanying drawings.

1. An apparatus to monitor for hypoglycaemia in a human, the apparatus including:
two or more sensors to concurrently measure two or more physical properties over time, the physical properties including one or more temperatures and/or one or more movements of an arm and/or a hand of the human; and
an electronic system configured to:
receive signals representative of the measured physical properties; and
process the received signals to generate an output indicative of hypoglycaemia in the human.

2. The apparatus of claim 1, wherein the sensors include at least one accelerometer and/or gyroscope to measure at least one corresponding tremor movement of the arm and/or of the hand.

3. The apparatus of claim 2, wherein the tremor movement of the arm and/or of the hand includes at least one tremor movement of a finger of the hand.

4. The apparatus of claim 2, wherein the at least one tremor movement of the arm and/or of the hand includes at least one tremor movement of a wrist of the arm.

5. The apparatus of claim 1 wherein the sensors include one or more reference temperature sensors to measure respective ambient temperatures of an environment around the human.

6. The apparatus of claim 1, including any one or more of:
an arm assembly with one or more of the sensors and an arm mount for mounting on the arm of the human; and
a finger assembly with one or more of the sensors and a finger mount for mounting on the hand of the human.

7. The apparatus of claim 1, including one or more pressure sensors to measure respective pressures of attachment between the apparatus and the arm and/or the hand.

8. The apparatus of claim 7, wherein the electronic system is configured to process a signal representative of the measured pressures of attachment to determine whether the apparatus is properly mounted to the human, and to generate an alarm signal if the apparatus is not properly mounted to the human.

9. The apparatus of claim 1, wherein the electronic system is configured to generate an alert if the output is indicative of hypoglycaemia.

10. The apparatus of claim 1, including at least one of a display, an audio transducer and a vibration transducer.

11. The apparatus of claim 1, wherein the apparatus is configured to send an alert signal to an external communicating device if the output is indicative of hypoglycaemia.

12. The apparatus of claim 1, wherein the electronic system is configured to control at least one connector to operate selectively in each of two states, including:
a first state in which the connector receives direct current (DC) power to charge a battery of the apparatus, and in which the connector transmits and receives communication data according to a first protocol; and
a second state in which the connector provides DC power from the battery, and in which the connector transmits and receives communication data according to a second protocol.

13. A method to monitor for hypoglycaemia in a human, the method including:
concurrently measuring two or more physical properties over time, the physical properties including one or more temperatures and/or one or more movements of an arm and/or a hand of the human;
receiving signals representative of the measured physical properties; and
processing the received signals to generate an output indicative of hypoglycaemia in the human.

14. The method of claim 13, wherein the one or more movements include at least one tremor movement of the arm and/or of the hand.

15. The method of claim 14, wherein the tremor movement includes at least one corresponding tremor movement of a finger of the hand and/or wherein the tremor movement includes at least one tremor movement of a wrist of the arm.

16. (canceled)

17. The method of claim 13, including measuring respective ambient temperatures of an environment around the human.

18. The method of claim 13, including any one or more of:
mounting one or more of the sensors on the arm; and
mounting one or more of the sensors on the hand.

19. The method of claim 13, including measuring one or more pressures of attachment between an apparatus including two or more sensors for the two or more physical properties and the arm and/or the hand.

20. The method of claim 19, including processing a signal representative of the measured pressures of attachment to determine whether the apparatus is properly mounted to the human, and to generate an alarm signal if the apparatus is not properly mounted to the human.

21. The method of claim 13, including generating an alert if the output is indicative of hypoglycaemia

22. The method of claim 13, including displaying a visual alert, generating an audible alert, and/or generating a vibration alert and/or including sending an alert signal if the output is indicative of hypoglycaemia, to an external communicating device.

23. (canceled)

24. The method of claim 13, including:
in a first state, receiving direct current (DC) power to charge a battery, and transmitting and receiving communication data according to a first protocol; and
in a second state, providing DC power from the battery, and transmitting and receiving communication data according to a second protocol;
wherein the communication data at least partially represent the signals.
